(19) United States
(12) Patent Application Publication
(10) Pub. No.: US 2009/0117073 A1
(43) Pub. Date: May 7, 2009

(54) USE OF A FUSION PROTEIN TARGETING THE ED-B FIBRONECTIN DOMAIN FOR TREATMENT OF ATHEROSCLEROSIS

(76) Inventors: Andreas Menrad, Ely Cambs (GB); Hans Dietrich Menssen, Berlin (DE); Kristof Graf, Kleinmachnow (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 11/928,692

(22) Filed: Oct. 30, 2007

Related U.S. Application Data
(60) Provisional application No. 60/857,170, filed on Nov. 7, 2006.

(30) Foreign Application Priority Data
Oct. 31, 2006 (EP) 06076951.0

(51) Int. Cl. A61K 38/20; A61K 38/02; A61K 39/395

(52) U.S. Cl. 424/85.2; 514/2; 424/134.1

(57) ABSTRACT

The present invention relates to the use of a fusion protein comprising an antibody part which specifically recognises ED-B fibronectin, an effector part and optionally one or more fusion protein linker(s) and/or antibody linker(s), for the manufacturing of medicaments for the treatment and prevention of atherosclerosis

This application claims the benefit of the filing date of U.S. Provisional Application Ser. No. 60/857,170 filed Nov. 11, 2006, which is incorporated by reference herein.

The present invention relates to the use of a fusion protein comprising an antibody part which specifically recognises the extra domain B (ED-B) of fibronectin, an effector part and optionally one or more fusion protein linker(s) and/or antibody linker(s), for the manufacturing of medicaments for the treatment and prevention of atherosclerosis.

Atherosclerosis is known as a chronic inflammatory lipid storage disease of large and medium-sized arteries complicated by cardiovascular events. These are commonly the result of sudden arterial thrombosis in the heart, brain, legs, and other organs. Pathologic intimal thickening as a result of phospholipids and cholesterol deposition constitutes the earliest detectable atherosclerotic change which is followed by macrophage and CD4+ and CD8+ T cell invasion (Virami R, et al., Arterioscler, Thromb Vasc Biol 2005, 25:2054-61; Xu Q B, et al., Clin Immunol Immunpathol, 1990, 56: 344-359). Subsequently, this process leads to proliferation of the vasa vasorum which perfuses the atherosclerotic plaques (Rose R. N Engl J. Med., 1999, 340:115-126; Wilson S H, et al., Circulation., 2002, 105:415-418). Rupture of the vasa vasorum resulting in intraplaque haemorrhage is an important process in the progression of asymptomatic plaques into high-risk unstable lesions (Kolodgie F D, et al., N Engl J. Med., 2003, 349:2316-2325). Intraplaque heamorrhage and plaque rupture are associated with an increased density of mircovessels (Fleiner M, et al., Circulation 2004, 110, 2843-2850). Plaque rupture is the principal cause of luminal thrombosis in acute coronary artery syndromes occurring in 75% of patients dying of acute myocardial infarction (Davies M J, et al., N Engl J Med, 1984, 310: 1137-1140). Methods for the effective imaging of said atherosclerotic plaques and methods of treatment and prevention thereof are of considerable interest. The prevention of coronary artery disease (CAD) or atherosclerosis in general involves therapeutic lifestyle changes such as smoking cessation, diet, weight reduction and exercise. In patients with established CAD or atherosclerosis in other vascular beds, or in patients at high risk of developing CAD, lowering serum total and low-density lipoprotein cholesterol (LDL-C) has been associated with a reduction in cardiovascular morbidity and mortality, and total mortality. Therapy with 3-hydroxy-3-methylglutaryl coenzyme A reductase inhibitors (such as atorvastatin, Lipitor®; pravastatin, Pravachol®; simvastatin, Zocor)) has had a major impact on preventive cardiology. Clinical trials have consistently shown that the reduction in serum cholesterol correlates with a decrease in major cardiovascular events, irrespective of the method used to reduce cholesterol. However, there is little trial-based evidence to support the early clinical data of these agents in patients with acute coronary syndromes. Usually, these patients are treated with platelet inhibitors (e.g. Aspirin® or clopidogrel, Plavix®), anticoagulants (sc heparin injections), and vaso-dilators (e.g. nitroglycerine, calcium antagonists). These systemic therapies may transiently reduce clinical symptoms in CAD patients, but they are not known for reducing the plaque size, improve or stabilize the plaque morphology, or having a striking early impact on progressive atherosclerosis. For manifest and or clinically symptomatic CAD, the current therapy of choice includes coronary balloon angioplasty and coronary artery bypass graft surgery (Braunwald E, et al., in Harrison's Principle of Internal Medicine, most recent edition (2005), McGraw-Hill).

Although these invasive therapies may result in relief of acute or subacute symptoms, since local in nature, they have no major impact on the underlying progressive atherosclerotic disease.

One of the most selective markers associated with angiogenesis known so far represents the extra domain B (ED-B) fibronectin. FNs are high molecular-mass extracellular matrix (ECM) components abundantly expressed in a range of healthy tissues and body fluids. Various different FN isoforms can be generated due to alternative splicing at the level of the primary transcript. The ED-B, a small domain of 91 amino acids, which is identical in sequence in mouse, rat and man, is usually absent in both plasma and tissue-fibronectin, except for some blood vessels of the regenerating endometrium and the ovaries (Alessi P. et al., Biochim. Biophys. Acta, 2004, 1654: 39-49; Viti F. et al., Cancer Res., 1999, 59: 347-352). However, it may become inserted into the fibronectin molecule during active tissue remodeling associated with neo-angiogenesis, thereby specifically accumulating around neovascular structures, such as around the neo-vasculature in atherosclerotic plaques. Thus, the ED-B fibronectin, and in particular the ED-B domain as such, represents a target for molecular intervention (Zardi et al., EMBO J., 1987, 6: 2337-2342, Carnemolla et al., J. Cell Biol., 1989, 108: 1139-1148, Castellani et al., Int. J. Cancer, 1994, 59: 612-618).

In this context, molecules capable of selectively targeting markers of angiogenesis create clinical opportunities for the diagnosis and therapy of diseases characterised by vascular proliferation, such as rheumatoid arthritis, diabetic retinopathy and age-related macular degeneration (O'Reilly et al., Nat. Med., 1996, 2: 689, O'Reilly et al., Cell, 1997, 88: 277, Friedlander et al., Science, 1995, 270: 1500, Pasqualini et al., Nat. Biotechnol., 1997, 15: 542, Huang et al., Science, 1997, 275: 547, Kim et al., Nature, 1993, 362: 841, Schmidt-Erfurth et al., Br. J. Cancer, 1997, 75: 54).

Recently, a number of good quality antibodies specific for the ED-B of fibronectin have been generated. In particular, the human single chain Fv antibody fragment L19, which displays a picomolar binding affinity for ED-B, has been verified to selectively target newly formed tumor blood vessels, e.g. in experimental tumor models (Viti F. et al., Cancer Res., 1999, 59: 347-352; Tarli L, et al., Blood, 1999, 94: 192-198) and tumor lesions in patients with solid cancers (Santimaria M. et al., Clin. Cancer Res., 2003, 9: 571-579). Other antibodies which may be used according to the invention are BC-1, CGS-1 and CGS-2. BC-1 specifically recognizes ED-B fibronectin, but does not bind to the ED-B domain of ED-B fibronectin.

Cytokines are a group of proteinaceous signaling compounds that are used extensively for inter-cell communication. Apart from their importance in the development and functioning of the immune system, cytokines also play a major role in a large number of immunological, inflammatory and infectious diseases. It has been shown in the past that cytokines can be very potent compounds for the treatment of disorders in the human and animal body. Interferon-alpha is active as a monotherapy against chronic myeloid leukemia (CML) and hairy cell leukemia and can induce complete long-lasting remissions in some patients (Kamtarjian H M, et al., Blood 2006, 108:1835-1840; Baker P K, et al., Blood 2002, 100:647-653; Damasio E E, et al., Eur J Haematol 2000, 64: 42-52). Pegylated Interferon-alpha is widely used as an active treatment, alone or in combination with lamivudine or adefovir, against chronic hepatitis B, C, and D (Wursthom K, et al., Hepatology 2006, 44: 675-684; Desmond, C P, et al., J Viral Hepatitis 2006, 13: 311-315; Niro G A, et al., Hepatology 2006, 44:713-720). Interferon beta is widely used as a treatment for multiple sclerosis (Kappos L, et al., Neurology 2006, 67:944-953). Tumour necrosis factor alpha (TNFα) is approved in combination with melphalan as a limb-sparing therapy for sarcoma patients in the context of isolated limb perfusion (ILP; Grunhagen D, et al., Cancer 2006, 106:1776-84). It is also successfully used in the same setting for melanoma patients (Lejeune F, et al., Cancer Immunity, 2006, 6: 1-17).

Particularly, interleukin-2 (IL2) has been characterized as one of the most potent cytokines, especially in anti-tumor experiments. It exhibits panoply of immune regulatory effects, including the stimulation of various anti-tumor effector cells. (Rosenberg S. A., J. Intern. Med., 2001, 250: 462-475). However, despite being approved for the clinical treatment of metastatic renal cell carcinoma, systemically applied IL2 has not been proven as successful as one had hoped. Therapeutic efficacy of systemically applied IL2 is thwarted by its serious, potentially life-threatening side effects (e.g. orthostatic hypotension, vascular leak syndrome and profound malaise) that limit dose escalation and prevent the administration of a curative dose (Bubenik J. et al., Cancer Immunol. Immunother. 2000, 49: 116-122; Baluna R. et al., Proc. Natl. Acad. Sci. USA, 1999, 96: 3957-3962). Additionally, the rapid degradation or elimination of IL2 delivered systemically further decreases its effectiveness. On the other hand, local administration of IL2 has been more successful and has resulted in the control of malignant effusions and the generation of significant remission of established tumour lesions (Bubenik J. et al., Cancer Immunol. Immunother., 2000, 49: 116-122; Den Otter W. et al., J. Urol., 1998, 159: 1183-1186; Baselmans A. H. et al., Cancer Immunol. Immunother., 2002, 51: 492-498; Krastev Z. et al., Hepatogastroenterology, 2003, 50: 1647-1649, Radny P, et al., Br J Cancer 2003, 89:1620-1626).

IL2 is critical for the expansion and regulation of regulatory T cells (T_{reg}), also termed CD4⁺CD25⁺T Cells (Thornton A. M. et al, J. Immunol., 2004, 172:6519-6523). The IL2/IL2 receptor pathway is elementary for a competent immune system, since genetic deletion of one member of the pathway such as IL2, IL2Rα or ILRβ leads to early death in mice by severe lymphoproliferation and autoimmune disease. Generally, T_{reg} are important to suppress T cell proliferation in vitro, and suppress immune response to auto- and allo-antigens, tumor antigens and infectious agents in vivo (Shevach, E. M., 2002, Nat. Rev. Immunol. 2:389). T_{reg} play a role in the development of atherosclerosis (Ait-Oufella, H. et al., Nat. Med., 2006, 12:178). Matter et al. have for example described imaging methods for displaying atherosclerotic plaques in ApoE−/− mice (atherosclerosis model) using an fibronectin ED-B-specific antibody, which has been labelled with a radioactive or infrared-sensitive marker (cf. Matter C. M. et al., Circulation Research, 2004, 24: 1225-1233). However, the vague therapeutic outlook proposed by Matter et al., according to which human monoclonal antibody L19 fused with interleukin-12 (IL12), among other L19-based fusion proteins, might eventually be therapeutically effective against said atherosclerotic plaques, does not match up with the scientific findings of others. (cf. Davenport P. et al., Am. J. Pathology, 2003, 20: 264-269; Zhao L. et al., J B C, 2002, 277: 35350-35356; Lee T. S. et al., Atheroscler. Throm. Vasc. Biol., 1999, 19: 737-742; Zhou R. H. et al., J. Atheroscler. Thromb., 2001, 8: 30-32; Hauer A. D. et al., Circulation, 2005, 16: 1054-1062). Accordingly, presence of IL12 in atherosclerotic plaques has been identified as a key inducer of a type 1 T-helper cell cytokine pattern, which is thought—in striking contrast to the suggestion of Matter et al., —to contribute to the development of atherosclerosis, rather than to prevent or treat it. In particular, Davenport et al. have shown that IL12-knockout in ApoE−/− mice leads to a significant reduction in plaque formation. This result is supported Zhao et al., who used targeted gene disruption or overexpression of 12/15-lipoxygenase in mice (background of apolipoprotein E or low density lipoprotein-receptor deficiency) and found a 50% decrease in aortic lesions at 8 months in mice on chow diet (no cholesterol difference). In the cultured macrophages of these mice they discovered a remarkable 75-90% decrease in IL12 production. Lee et al. found IL12 expression in macrophages of aortic plaques of apo-E-deficient mice, and that daily applications of IL12 to these mice accelerated atherosclerosis. Similarly, Zhou R. H. et al. have found elevated serum IL-12 levels in patients with acute myocardial infarction but not in patients with unstable angina pectoris, suggesting that IL12 is involved in the progression of CAD. Finally, these findings prompted Hauer et al. to design a vaccination technique in mice that fully blocks the biologic action of IL12. Vaccination of LDL receptor-deficient mice (LDLr−/−; another atherosclerosis animal model) against the biologic activity of IL12 resulted in a significant 68.5% reduction of artherogenesis.

The publication by Subramanya Upadhya et al. (2004; Atherogenic effect of Interleukin-2 and antiatherogenic effect of Interleukin-2 antibody in Apo-E-Deficient Mice, Angiology, Vol. 55 No. 3, pages 289-294) demonstrates a pro-atherosclerotic effect caused by interleukin-2 enhancing atherosclerosis. Further, anti-IL-2 antibodies have a protective effect against atherosclerosis.

In view of the above, an urgent need exists for compositions and/or medicaments which can be used in the specific treatment of said atherosclerotic plaques, and according to the scientific data stated, IL12 or targeted IL12 obviously cannot be considered a possible solution to this therapeutic problem.

Thus, the technical problem underlying the present invention is to overcome the above-mentioned problems by providing novel medicaments for the specific treatment and prevention of atherosclerotic plaques in humans and animals and other diseases connected thereto.

The problem is solved by the embodiments as described in the claims and the description.

The invention relates to the use of a fusion protein comprising at least one targeting part and at least one effector part, wherein the targeting part specifically recognises ED-B fibronectin and wherein the effector part is a cytokine or a biologically active fragment thereof, in the manufacture of a medicament for the treatment and prevention of atherosclerosis.

In a preferred embodiment, the targeting part binds to the extra domain B (ED-B) of fibronectin.

In particular, there is provided the use of a fusion protein comprising at least one targeting part and at least one effector part, wherein the targeting part specifically recognises the extra domain B (ED-B) of fibronectin and wherein the effector part is a cytokine or a biologically active fragment thereof, in the manufacture of a medicament for the treatment and prevention of atherosclerosis.

The term “fusion protein” used herein relates to an artificial proteinaceous construct and means a protein comprising at least two different amino acid sequences which are defined by their origin and/or by special functions. Moreover, the term fusion protein according to the present invention does further include such fusion proteins which also contain non-protein molecule parts such as nucleic acids, sugars, or markers for radioactive or fluorescent labelling.

Further, the term “targeting part” used according to the present invention means any molecule or group of molecules which selectively binds to at least a portion of a desired target molecule, such as a receptor, an antigen or fragments thereof. Examples of said targeting part include hormones, neurotransmitters, antibodies and fragments thereof and antibody mimetics.

As used herein, the expressions “fragment” or “biologically active fragment” mean a part or a combination of parts of a molecule or group of molecules, as long as the desired effects, such as targeting activity or biological and pharmaceutical activity, are maintained.

According to a preferred embodiment of the present invention, in the use as defined above, the targeting part is an antibody or a fragment thereof.

The term “antibody” used in the present invention is not specifically limited and includes for example full-length antibodies, native antibodies, monoclonal antibodies, chimeric antibodies, humanized antibodies, and human antibodies, full IgG antibodies, multispecific antibodies (e.g. bispecific antibodies) formed from at least two intact antibodies.

Said term “antibody” is further used in the broadest sense and specifically covers intact monoclonal antibodies, polyclonal antibodies, multispecific antibodies (e.g. bispecific antibodies) formed from at least two intact antibodies, and antibody fragments thereof as long as they exhibit the desired biological targeting activity.

The term “antibody fragments” used herein comprises a portion of an intact antibody, preferably comprising the antigen-binding or variable region thereof or CDR regions thereof, as long as they exhibit the desired biological targeting activity.

Examples of such antibody fragments include Fab, Fab′, F(ab′)2 and Fv fragments, diabodies, minibodies, linear antibodies, single-chain antibody molecules, small immuno-proteins (SIPs), “scFv”, and multispecific and multivalent antibodies formed from antibody fragments.

According to a particularly preferred embodiment, the targeting part is the human recombinant antibody L19 or a fragment or derivative thereof.

According to another embodiment, the targeting part is the antibody BC-1 or a fragment or derivative thereof.

According to preferred embodiment, the L19 antibody or antibody fragment is in scFv, SIP, IgG or Fab format, preferably in scFv format.

The antibodies or antibody fragments can be in monomeric or multimeric, for example dimeric form. Multimeric forms may be homomeric or heteromeric. The multimeric forms may be formed by covalent linkage or by non-covalent association. For example, L19-IL2 in scFv format forms noncovalent homodimers. Another example is L19-SIP, which forms covalent, homomeric dimers.

In particular, the antibody or antibody fragment comprises the CDR regions of L19 and/or comprises at least one Vh and at least one Vl chain of the L19 antibody.

In a preferred embodiment of the present invention, the heavy chain of the L19 antibody or antibody fragment has a sequence according to SEQ ID NO: 1 and/or the light chain of said antibody has a sequence according to SEQ ID NO: 2.

In another preferred embodiment, the antibody or antibody fragment comprises the CDR regions of L19 antibody or antibody fragment. In particular, the CDR sequences of L19 are shown in SEQ ID 8 to 13.

According to another embodiment, in the use as defined above, the heavy and the light chain of the antibody or antibody fragment are connected by an antibody linker.

The term “antibody linker” as used herein is not especially restricted and may be any antibody linker known in the art, such as an amino acid, a peptide, or an aliphatic or aromatic organic molecule. Examples of such linkers are described in EP 0 573 551, EP 0 623 679 and EP 0 318 554.

In a specific example of the above-defined use, the antibody linker has a sequence according to SEQ ID NO: 3.

“Antibody mimetics” are understood as binding molecules based on protein frameworks (“scaffolds”) which specifically bind to the target and which are distinct from antibodies and antibody fragments. Such scaffolds are described in Binz et al., 2005, Nat. Biotechnol. 23, 1257-1268. Antibody mimetics specifically binding to ED-B fibronectin are described in Grabulovski et al., J. Biol. Chem., 2007, 282:3196-3204.

According to the present invention, the term “effector part” is not specifically restricted and means any cytokine known in the art, excluding interleukin-12 (IL12). Examples of such cytokines are interleukins (IL) such as IL1α and IL1β, IL2 to IL11 or IL 13 to IL22, as well as interferons (IFN), such as IFN-α, IFN-β or IFN-γ, and Tumour Necrosis Factor alpha (TNFα).

In a further embodiment of the above-defined use present invention, the cytokine is an interleukin or a biologically active fragment or derivatives thereof, excluding IL12.

According to a preferred embodiment of the present invention, the cytokine in the above-defined use is interleukin-2 (IL2) or a fragment or derivative thereof. Preferably, the interleukin-2 is human.

In another embodiment of the present invention, the cytokine in the above-defined use is Tumour Necrosis Factor alpha (TNFα) or a fragment or derivative thereof. Preferably, the TNFα is human.

According to one example of the present invention, the interleukin-2 has a sequence according to SEQ ID NO: 4.

According to one example of the present invention, the TNF alpha has a sequence according to SEQ ID NO: 6 or SEQ ID NO: 7.

According to a preferred embodiment of the present invention, the fusion protein contains L19-IL2 and has a sequence according to the addition of the sequences SEQ ID NO: 1, SEQ ID NO: 3, SEQ ID NO: 2, SEQ ID NO: 5 and SEQ ID NO: 4. (in the direction from the N terminal to the C terminal).

In another embodiment, the fusion protein L19-TNFalpha comprises the following succession of elements: SEQ ID NO: 1, SEQ ID NO: 3, SEQ ID NO: 2, SEQ ID NO: 5 and SEQ ID NO: 6 (in the direction from the N terminal to the C terminal). In another embodiment, the fusion protein L19-TNF alpha comprises the following succession of elements: SEQ ID NO: 1, SEQ ID NO: 3, SEQ ID NO: 2, SEQ ID NO: 5 and SEQ ID NO: 7 (in the direction from the N terminal to the C terminal).

In one embodiment of the present invention, the fusion protein in the above-defined use has (a) an N-terminal targeting part and a C-terminal effector part, or (b) an N-terminal effector part and a C-terminal targeting part.

According to another embodiment of the present invention, the targeting part specifically binds to oncofetal ED-B fibronectin.

In another embodiment of the above-defined use, the targeting part and the effector part are connected by a fusion protein linker. A preferred embodiment of this fusion protein linker is described in SEQ ID NO 5.

The term “fusion protein linker” used herein is not specifically restricted and may include any linker usable to connect the targeting part and the effector part according to the present invention, such as an amino acid, a peptide or an aliphatic or aromatic organic molecule. Specific examples of fusion protein linkers are described in EP 0 573 551, EP 0 623 679 and EP 0 318 554.

Another aspect of the present invention relates to the method of treating atherosclerotic plaques and the diseases connected thereto in a patient, comprising the step of administering a fusion protein according to the present invention to said patient.

The fusion protein according to the present invention may be administered typically in combination with one or more auxiliary agents, such as pharmaceutically acceptable carriers, buffers or salts.

The route of administration of the composition of the present invention does not exhibit a specific limitation and can be, for example, subcutaneous or intravenous. Preferred is the intravenous application and the subcutaneous application. The term “patient” as used in the present invention includes mammals, particularly humans.

The fusion protein with IL2 according to the present invention may be administered in an amount of about 0.5 to 60 Mio IU IL2 equivalent (corresponding to 0.835 to 10.02 mg fusion protein) per application and human patients, preferably 5 to 60 Mio IU IL2 equivalent (corresponding to 0.835 to 10.02 mg fusion protein) per application and human patients. Treatment might be given in a repeated fashion parenterally either iv or sc. (e.g. on day 1, 3, and 5, repeat on day 22 or possibly daily). Different application schemes might be necessary to obtain full clinical benefit. Prevention can be achieved by application in a comparable manner to humans in risk of developing atherosclerosis.

The figures show:

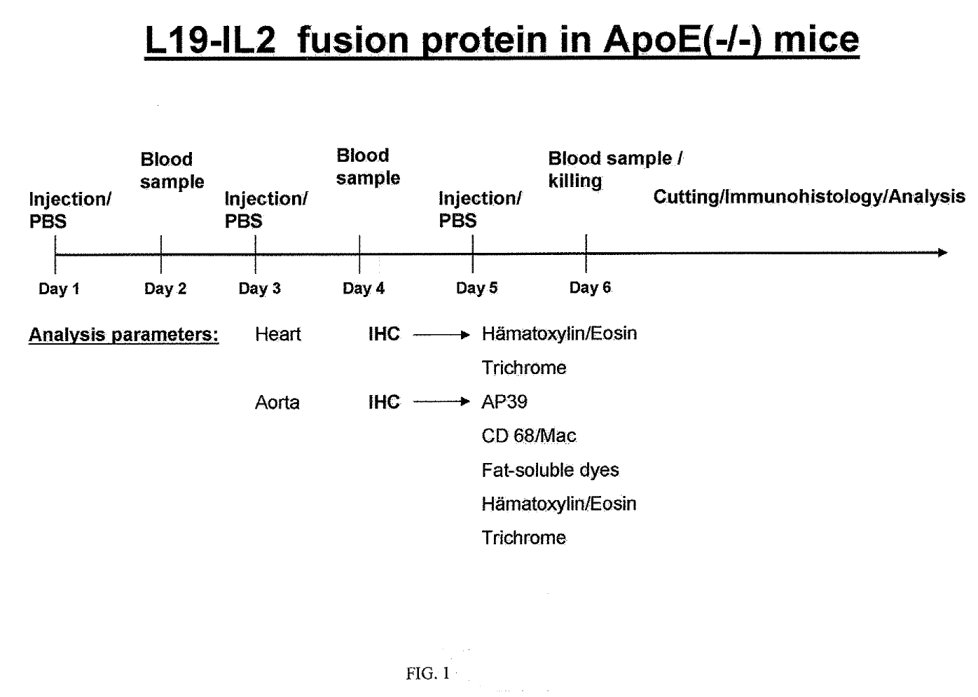

FIG. 1 shows the schematic course of the L19-IL2 fusion protein experiments in ApoE(−/−) mice.

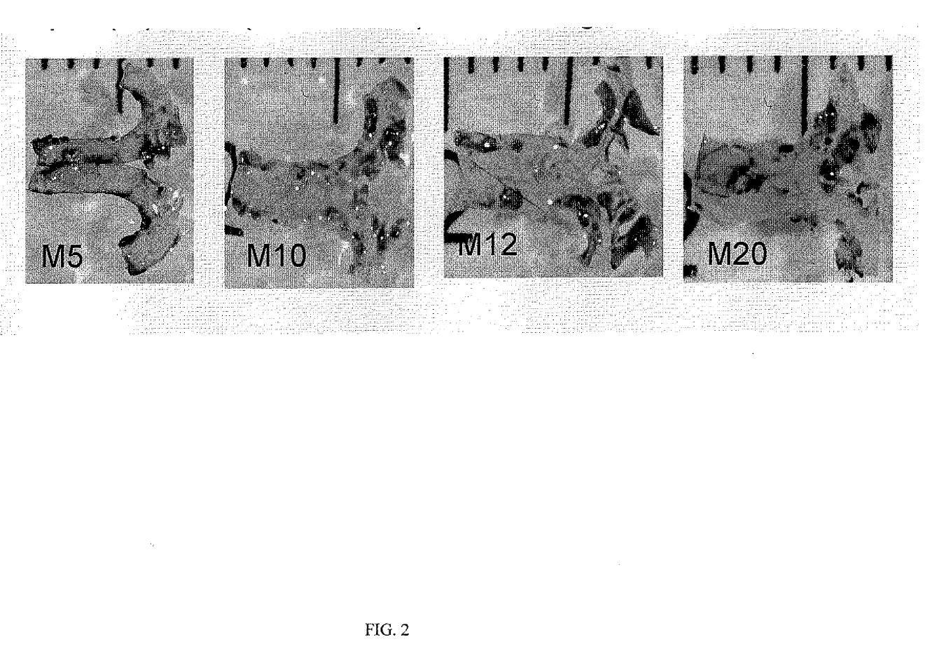

FIG. 2 shows photographs of thoracic aortas prepared from ApoE(−/−) mice. The dark regions indicate fatty lesions visualized using Sudan dyes. These lesions correspond to atherosclerotic plaques.

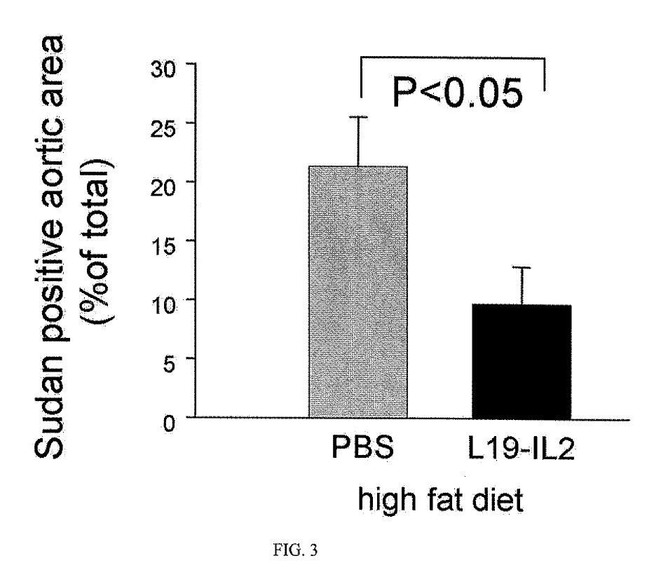

FIG. 3 shows a diagram on the effect of L19-IL2 fusion protein on atherosclerotic plaque formation in thoracic aortas of ApoE(−/−) mice at an age of 5 months and fed with a high fat diet.

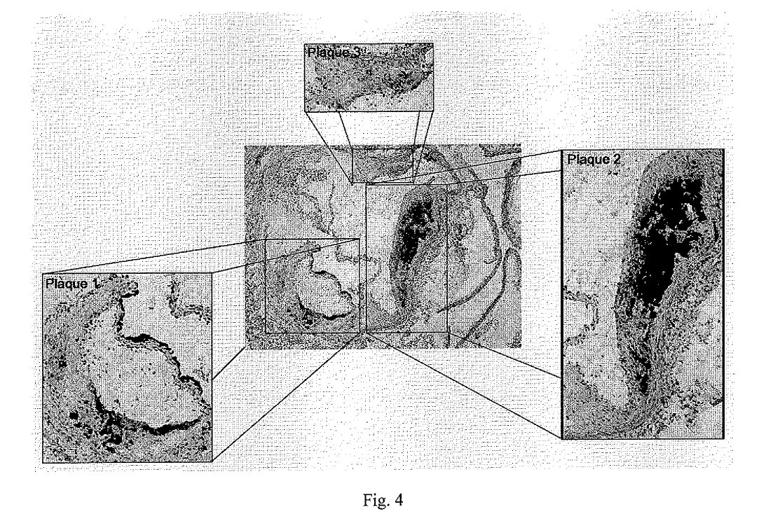

FIG. 4 shows the photographs of histo-morphology of ED-B in atherosclerotic plaques of the aortic root of 6-months-old apoE(−/−) mice fed with normal chow (normal diet). Dark areas indicate high accumulation of the ED-B of fibronectin binder especially around the vasa vasorum of the plaques and in the endothelial linings of the plaque cap.

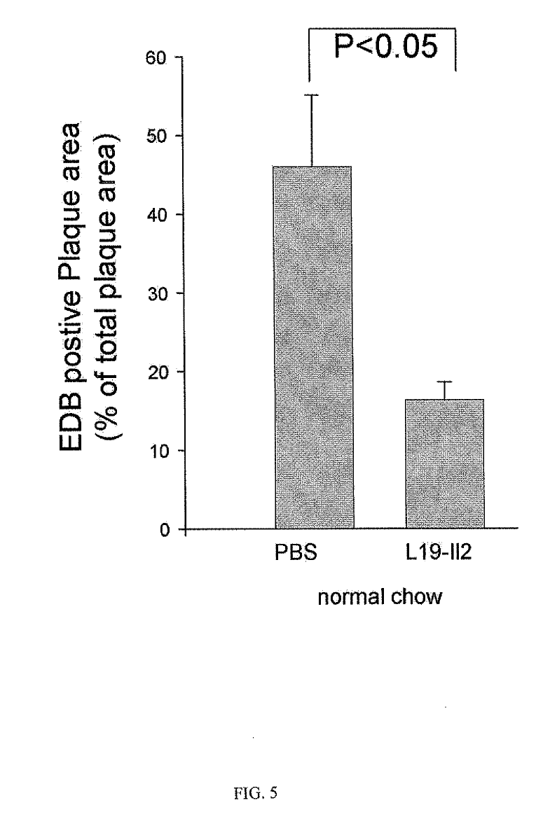

FIG. 5 shows the effect of L19-IL2 on formation of ED-B positive plaque area in the aortic root of ApoE(−/−) mice (6 month old) fed with normal fat diet.

The present invention provides a fusion protein which comprises at least one targeting part and at least one effector part, wherein the targeting part specifically recognises the ED-B fibronectin, in particular binds to the extra domain B (ED-B) of fibronectin, in the manufacture of a medicament for the treatment and prevention of atherosclerosis. Since ED-B expression can only be found in very few tissues and/or locations in the human and animal body, such as in atherosclerotic plaques, and since it is not expressed in the majority of healthy mature human organs, the use according to the present invention advantageously allows for the production of highly specific medicaments which target these tissues and/or locations. Thus, through the use of the fusion protein according to the present invention, the obtained medicaments enable the transport of the effector part to the desired tissue or location. Due to the surprisingly high specificity of the fusion protein for the ED-B of fibronectin, a medicament is provided that allows, by use of a suitable effector part, such as the cytokine IL2 or TNFα, in particular IL2, the direct and highly efficient treatment of the targeted tissue and/or location.

The targeted concentration of a cytokine such as IL2 in the atherosclerotic plaque microenvironment by conjugating it for example to the homodimeric scFv L19 antibody, specific for the ED-B of fibronectin, enhances the therapeutic index of the cytokine and at the same time diminishes its toxic side effects, thereby providing a valuable therapeutic tool for the treatment and prevention of atherosclerotic plaques and further diseases connected thereto.

### EXAMPLES

The present invention will be further illustrated in the following examples, without any limitation thereto.

### Example 1

### Treatment of ApoE Mice with Fusion Protein L19-IL2

For evaluation of the progression of atherosclerosis, myocardial events and mortality 6 to 7 months old ApoE(−/−) mice are fed with normal chow and high fat diet. The fusion protein L19-IL2 is applied 3 times/week in sterili phosphate buffered saline via i.v. injection at a dose of 10⁶ IU/kg IL2 equivalents. Blood was collected by retro-orbital bleeding. A diagram showing the experimental course is given in FIG. 1.

The result of these experiments show that is was surprisingly found that the addition of L19-IL2 leads to reduced plaque formation (FIG. 3)

### Example 2

### Histology of the Heart

For determining the impact of the fusion protein L19-IL2 on the histology of the heart, both hearts of L19-IL2-treated animals and of control animals are analysed for areas of infarction, infiltration of mononuclear cells and obliterated coronary arteries. For this purpose, tissue samples were snap-frozen in liquid nitrogen, embedded in OCT and stored at −80° C. for immunohistochemistry. For this purpose, 5-10 μM tissue sections were fixed and stained with the respective antibodies according to standard immunohistochemical procedures.

The histological experiments demonstrate that no significant differences between control and L19-IL2-treated animals occur.

### Example 3

### Serum Parameters

According to these serologic experiments both control- and L19-IL2 groups show no differences in troponin levels (a highly sensitive and specific indicator of myocardial infarction).

Without further elaboration, it is believed that one skilled in the art can, using the preceding description, utilize the present invention to its fullest extent. The preceding preferred specific embodiments are, therefore, to be construed as merely illustrative, and not limitative of the remainder of the disclosure in any way whatsoever.

In the foregoing and in the examples, all temperatures are set forth uncorrected in degrees Celsius and, all parts and percentages are by weight, unless otherwise indicated.

The entire disclosures of all applications, patents and publications, cited herein and of corresponding European application No. 06076951.0, filed Oct. 31, 2006, and U.S. Provisional Application Ser. No. 60/857,170 filed Nov. 11, 2006, are incorporated by reference herein.

The preceding examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions.

1. A method for the treatment and/or prevention of atherosclerosis comprising administering a fusion protein comprising at least one targeting part and at least one effector part, wherein the targeting part specifically recognises ED-B fibronectin and wherein the effector part is a cytokine or a biologically active fragment thereof.

2. The method according to claim 1, wherein the targeting part binds to the extra domain B (ED-B) of fibronectin.

3. The method according to claim 1, wherein the targeting part is an antibody or a biologically active fragment thereof.

4. The method according to claim 2, wherein the targeting part is the human recombinant antibody L19 or a biologically active fragment thereof.

5. The method according to claim 3, wherein the antibody or antibody fragment comprises the CDR regions of L19 and/or comprise at least one Vh and at least one Vl chain of the L19-antibody.

6. The method according to claim 2, wherein the heavy and the light chain of the antibody are connected by an antibody linker.

7. The method according to claim 2, wherein the antibody linker has a sequence according to SEQ ID NO: 3.

8. The method according to claim 1, wherein the cytokine is an interleukin or a biologically active fragment thereof.

9. The method according to claim 8, wherein the cytokine is interleukin-2 (IL2) or a biologically active fragment thereof.

10. The method according to claim 9, wherein the interleukin-2 has a sequence according to SEQ ID NO: 4.

11. The method according to claim 1, wherein the fusion protein has
(a) an N-terminal targeting part and a C-terminal effector part, or
(b) an N-terminal effector part and a C-terminal targeting part.

12. The method according to claim 1, wherein the targeting part and the effector part are connected by a fusion protein linker.

13. The method according to claim 12, wherein the fusion protein linker has a sequence according to SEQ ID NO: 5.


SEQUENCE LISTING (13 sequences)

SEQ ID NO: 1 - protein, 116 aa, Artificial Sequence [sequence omitted]

SEQ ID NO: 2 - protein, 108 aa, Artificial Sequence [sequence omitted]

SEQ ID NO: 3 - protein, 12 aa, Artificial Sequence
  Antibody linker
GDGSSGGSGGAS

SEQ ID NO: 4 - protein, 133 aa, Homo sapiens [sequence omitted]

SEQ ID NO: 5 - protein, 17 aa, Artificial Sequence
  Fusion protein linker
EFSSSSGSSSSGSSSSG

SEQ ID NO: 6 - protein, 155 aa, Homo sapiens
SSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIAL

SEQ ID NO: 7 - protein, 157 aa, Homo sapiens [sequence omitted]

SEQ ID NO: 8 - protein, 5 aa, Artificial Sequence
  CDR1 Vh
SFSMS

SEQ ID NO: 9 - protein, 17 aa, Artificial Sequence
  CDR2 Vh
SISGSSGTTYYADSVKG

SEQ ID NO: 10 - protein, 7 aa, Artificial Sequence
  CDR3 Vh
PFPYFDY

SEQ ID NO: 11 - protein, 12 aa, Artificial Sequence
  CDR1 Vl
RASQSVSSSFLA

SEQ ID NO: 12 - protein, 7 aa, Artificial Sequence
  CDR2 Vh
YASSRAT

SEQ ID NO: 13 - protein, 9 aa, Artificial Sequence
  CDR3 Vl
QQTGRIPPT